(19) United States
(12) Plant Patent Application Publication
(10) Pub. No.: US 2002/0092047 P1
(43) Pub. Date: Jul. 11, 2002

(54) Rose plant named 'Tananaistrua'

(75) Inventor: Hans-Jurgen Evers, Uetersen (DE)

Correspondence Address: Russell D. Orkin, 700 Koppers Building, 436 Seventh Avenue, Pittsburgh, PA, 15219-1818, US

(73) Assignee: Rosen Tantau Mathias Tantau Nachfolger

(21) Appl. No.: 09/758,720

(22) Filed: Jan. 11, 2001

(51) Int. Cl. A01H 5/00

(52) U.S. Cl. PLT/108

(57) ABSTRACT

The new cultivar is a variety of bed rose plant having an upright bushy growth habit with shiny red flowers. The plant exhibits good winter hardiness, resistance to most diseases, and excellent ground covering ability.

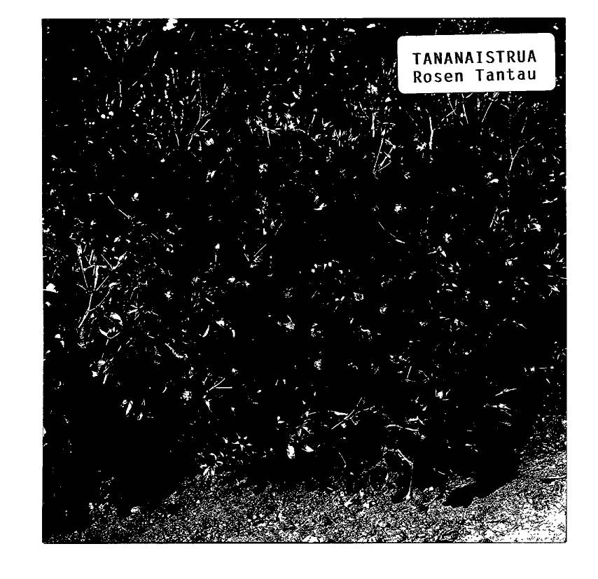

### BACKGROUND OF THE NEW PLANT

The present invention includes a new and distinct cultivar of Rosa l. known by the varietal name ‘Tananaistrua’.

The plant was discovered in 1991 by the breeder in Ueterson, Germany in a selective breeding program designed to produce a hardy, shiny red bed rose plant with good distance display and which is resistant to fungal diseases. The new plant is a selection from a cross of unnamed red floribunda seedlings maintained proprietary by the assignee of the present application. The plant was first asexually reproduced in 1996 and can be propagated asexually on Rosa canina rootstock.

The variety is distinguishable by its shiny red display, very good winter hardiness, disease resistance, and ground covering ability. Continued observations from the vegetative cuttings in Uetersen, Germany have confirmed that the distinguishing features of this new cultivar come true, remain stable, and are retained through successive propagations.

### DESCRIPTION OF THE DRAWING

The accompanying photographic drawing illustrates the new cultivar. Colors are described in part in reference to the R.H.S. Colour Chart of The Royal Horticultural Society of London.

### DESCRIPTION OF THE NEW PLANT

The following description sets forth the characteristics of the new cultivar.

The plant:

Botanical name.—Rosa l.

Size:

Height.—70 cm. Width: 50-60 cm.

Habit.—Upright, bushy.

Growth vigor.—Average.

Wood color.—Green.

Thorns.—Color: Red-brown. Size: 0.2 cm. Shape: Elongated.

Location on stems and branches: Random arrangement.

Disease resistance: Robust to fungi diseases, especially black spot and powdery mildew.

Hardiness: Robust to winter killing.

### FOLIAGE

Arrangement: Compound; unpaired pinnate.

Number of leaflets on typical stem leaves: 5.

Leaflets:

Size.—4-8 cm.

Color.—Upper surface: Dark green. Lower surface: Bright green.

Shape.—Elliptical.

Texture.—Smooth.

Margin.—Serrated.

Petiole rachis:

Stipules.—Size: 0.9 cm. Shape: Elongated.

### BUD

Size: 3 cm.

Shape: Bowl-shaped.

Color: Red.

Sepals:

Color.—Green.

Texture.—Smooth.

Receptacle:

Color.—Greenish-white.

Shape.—Circular.

Texture.—Funnel-shaped.

### FLOWER

Borne (number in clusters): 2.

Size (diameter): 11.5 cm of a cluster 7.5 cm of a fully open blossom.

Form: Circular.

Petals:

Number per flower. —13.

Color.—Upper surface: Red Groups 44B/43A. Lower surface: Red Groups 44B/43A.

Size.—3.5 cm.

Shape.—Nearly circular.

Persistence.—Maturing neatly.

Lastingness.—Long.

Fragrance: None.

Blooming period: Late June until early December in middle European climate.

### REPRODUCTIVE ORGANS

Stamens:

Number.—35.

Anther.—Length: 0.1 cm. Color: Brown.

Filament.—Length: 1 cm. Color: Yellow. Pollen color: Brown.

Pistils:

Style.—White-green in color.

Stigma.—Yellow in color.

I claim:

1. A new and distinct variety of rose plant substantially as shown and described.
